(19) United States
(12) Patent Application Publication
(10) Pub. No.: US 2003/0199487 A1
(43) Pub. Date: Oct. 23, 2003

(54) Ester and ether derivatives of 4-hydroxytestosterone and a method for the regulation of athletic function in humans

(75) Inventor: Sal Abraham, Dunmore, PA (US)

Correspondence Address: SAL ABRAHAM, 1304 ELECTRIC ST., DUNMORE, PA, 18509, US

(73) Assignees: Mr. Vince Paternoster; Nutri Sport Pharmacal, Inc., Franklin, NJ (US)

(21) Appl. No.: 10/064,027

(22) Filed: Jun. 4, 2002

Related U.S. Application Data
(63) Continuation-in-part of application No. 10/063,415, filed on Apr. 22, 2002, now Pat. No. 6,586,417.

(51) Int. Cl. A61K 31/58; A61K 31/56

(52) U.S. Cl. 514/178; 514/172

(57) ABSTRACT

This invention relates to a method of administering an effective amount of at least one of the ester or ether derivatives of 4-hydroxytestosterone for the regulation of athletic function in humans.

### BACKGROUND OF THE INVENTION

This is a continuation in part of Ser. No. 10/063,415, Filed on Apr. 22, 2001 (pending). This invention relates to the ester and ether derivatives of 4-hydroxytestosterone as a means of regulating athletic function in humans. In men, the normal balance of sex steroids is characterized by a greater amount of androgens over estrogens. When estrogen exceeds androgen levels in males a cascade of detrimental effects can take place for instance, decreased sperm count, low free testosterone levels, decreased muscle mass and decreased strength. When androgens are manipulated to exceed estrogen above the normal androgen to estrogen ratio the exact opposite takes place with an emphasis on the promotion of lean body mass and strength to aid in enhancing physical performance.

Serious and/or professional athletes are known to utilize intramuscular injection and/or peroral administration of pharmaceutical androgens for the promotion of muscle mass and athletic performance. Various testosterone esters in oil depot form have been utilized as intramuscular injection. These weekly injections of synthetic testosterone cause supraphysiological surges in androgen levels which then leave the body deficient in androgens until the next injection. These supraphysiological levels of testosterones readily convert to estrogen and dihydrotestosterone (DHT) which can lead to side effects such as gynocomastia and benign prostrate hypertrophy (BPH). Another negative side effect is the possible down regulation of the hypothalamic pituitary axis resulting in loss of natural testosterone production.

The oral route of synthetic androgen administration consists of testosterone derivatives that are 17 alpha-alkylated for enhanced oral bioavailability (i.e. Methyltestosterone). This alkylation allows the steroids to withstand the 17 beta-hydroxyl oxidation in the liver. This appears to alleviate the dosing and blood hormone problems as compared to injections but it can cause undue stress to the liver and increase the possibility of hepatotoxicity.

U.S. Pat. No. 2,762,818 to Levy, et al. discloses a method for the synthetic manufacturing of various esters of 4-hydroxytestosterone and discloses a medical use for the treatment of various states of androgen deficiency due to 4-hydroxytestosterone's anabolic and androgenic properties. The medical treatment referred to in this patent deals with the pharmaceutical application of 4-hydroxytestosterone for specific disease states. As with most androgens, 4-hydroxytestosterone is noted to increase the retention of nitrogen in metabolic processes. The majority of the patent then discloses eleven different methods for the synthesis of various esters of 4-hydroxytestosterone.

U.S. Pat. No. 5,880,117 to Patrick Arnold, relates a method of using of using the oral precursor hormone 4-androstenediol as a means of increasing testosterone levels in humans. This hormone represents an improvement in standard therapies, since 4-androstenediol does not seem to be open to aromatase in its initial state. The possibility that this compound will convert to an estrogen over an androgen is chemically unlikely. The end product testosterone however is still readily aromatized. The compound suggested in this patent does offer advantages over standard therapies in that the compound in question avoids a direct path of estrogen conversion and provides a less toxic peroral route of administration. The target hormone of replacement may still be less than ideal due to it's conversion to estrogen and dihydrotestosterone (DHT).

U.S. Pat. No. 6,242,436 to William Llewellyn, relates a method of using the oral precursor hormones 5alpha-androstanediol or 5alpha-androstanedione as a means of increasing dihydrotestosterone levels in humans. Dihydrotestosterone (DHT) is a more potent form of testosterone, shown to be roughly three to four times more active in the human body in comparison. This hormone represents an improvement, since 5alpha-androstanediol or 5alpha-androstanedione are natural, non-toxic, and quickly metabolized to active form after oral administration and unable to be aromatized into estrogens due to their structure. The compound suggested in this patent does offer advantages over standard therapies in that the compound in question avoids a direct path of estrogen conversion and provides a less toxic peroral route of administration. However, dihydrotestosterone (DHT) is not the most ideal form of testosterone due to it's causative relationship with benign prostrate hypertrophy (BPH) and other androgenic side effects.

### SUMMARY OF INVENTION

The use of illicit synthetic exogenous testosterone for the promotion of muscle mass and work performance is well known throughout athletic circles. However these therapies, besides being illegal, result in the aromatization of the target hormone and put undue stress upon the liver. Dietary supplement manufactures have attempted to correct these problems by the use of pro-hormones for the promotion of physical performance. For instance, U.S. Pat. No. 5,880,117 and U.S. Pat. No. 6,242,436 attempt to correct some of these problems by the peroral administration of androgen precursors. Now while the active androgen precursor is unable to aromatize, the target hormone of U.S. Pat. No. 5,880,117 can produce substantial conversion to estrogen and dihydrotestosterone (DHT) while the target hormone of U.S. Pat. No. 6,242,436 is directly converted to dihydrotestosterone (DHT). The problem of the present invention is to provide a compound that gradually increases androgen levels for the promotion of fat free mass and athletic performance without causing DHT accumulation, hepatotoxicity, supraphysiological surges in androgen blood concentrations, and down regulation of the hypothalamic pituitary axis. According to the invention these problems are solved by the use of the naturally occurring metabolite 4-hydroxytestosterone. The use of this compound involves a method of imparting an anabolic/androgenic effect in humans for the promotion lean body mass and physical performance with none of the previous mentioned negative side effects. 4-hydroxytestosterone is a naturally occurring metabolite of 4-hydroxy-4-androstenedione that can be sold as a dietary supplement. The ester and ether analogs of this compound can also be sold as a dietary supplement as long as the ester and ether additions are utilized for increasing oral bioavailability.

### DETAILED DESCRIPTION

The chemical term 4-hydroxytestosterone refers to two isomers: 4alpha,17beta-Dihydroxy-4-androsten-3-one and 4beta,17beta-Dihydroxy-4-androsten-3-one. This invention concerns both isomer forms of 4-hydroxytestosterone. 4-hydroxytestosterone is a natural metabolite of 4-hydroxy 4-androstenedione which, is an analog or derivative of the naturally occurring precursor hormone 4-androstenedione. Viable chemical esters of this compound include methyl, ethyl, propyl, butyl, and cyclohexyl carbonate as well as, acetate, heptanoate, decanoate, hemisuccinate, and benzoate. Viable chemical ethers include tetrahydropyranyl (THP), cyclopentyl (CPT), cyclohexyl and tetrahydrofuranyl (THF) ethers. This invention concerns the esters and ethers of the two isomer forms of 4alpha,17beta-Dihydroxy4-androsten-3-one and 4beta,17beta-Dihydroxy-4-androsten-3-one. The previous examples of various esters and ethers are presented by way of illustration only. It should be understood that this invention is not construed as limited in scope by the details contained therein, as it is apparent to those skilled in the art that modifications in materials and methods can be made without deviating from the scope of the invention.

Animal research performed by Davies J H, Shearer R J, Rowlands M G, Poon J K, Houghton J, Jarman M, Dowsett M (Enzyme Inhib) 1992,6:141-7 demonstrated 4-hydroxytestosterones inhibition of the 5-alpha-reductase enzyme and its androgenic potency. Four compounds were tested which included 4-hydroxy-4-androstenedione and three of its metabolites. These metabolites consisted of 4-hydroxytestosterone, 3beta-17-dihydroxy-5-alpha-androstan-4-one(metabolite A), and 3-alpha-17-dihydroxy-5-beta-androstan-4-one(metabolite B). 4-hydroxy-4-androstenedione and 4-hydroxytestosterone both displayed a weak inhibition of the 5 alpha-reductase enzyme while metabolites A and B had no significant inhibitory activity. This represents the notion that that 4-hydroxy-4-androstenedione and 4-hydroxytestosterone have the ability to alter testosterone metabolism by inhibiting the formation of 5-alpha dihydrotestosterone (DHT). Androgenic potency was also evaluated for all of the previously mentioned compounds utilizing the rat prostrate androgen receptor measured as the relative binding affinity (RBA). 4-hydroxy-4-androstenedione (RBA=0.085) and metabolites A (RBA=0.485) and B (RBA=0.016) bound weakly to the androgen receptor while 4-hydroxytestosterone exhibited a significant relative binding affinity (RBA=75). This RBA is comparable to potent androgens such as mibolerone (RBA=100) and 5-alpha dihydrotestosterone (DHT) (RBA=66). However, this assay did not distinguish between agonist or antagonist receptors. This lack of distinction inhibits the determination between the amount of androgenic and anti-androgenic effect. Although, this does suggest the ability of 4-hydroxytestosterone to directly impart a mild androgenic effect, while simultaneously altering testosterone metabolism by inhibiting the formation of 5-alpha dihydrotestosterone (DHT) resulting in a balancing of total androgen accumulation.

Animal research performed by Gardi R, Falconi G, Pedrali C, Vitali R, Ercoli A (Steroids) May 1972 19:5 639-47 demonstrates the androgenic and myogenic activity of orally administered ether modified alkyl androstan-17beta-yl mixed acetyls. The chemical addition of ethers and esters are designed to enhance oral absorption and bioavailability. The chemical addition of various ethers to the hydroxyl portion of the molecule, such as tetrahydropyranyl, tetrahydrofuranyl, cyclopentyl, or cyclohexyl ether, enables the compound to become lipophilic or fat soluble. This lipophilicity allows the compound to be absorbed via the lymphatic system and bypass the first-pass through the liver allowing more of the active compound to enter the bloodstream. More specifically this research demonstrates the androgenic and myogenic activity of an ether modified version of 4-hydroxytestosterone.

4,17beta-Dihydroxy-4-androsten-3-one (1′-methoxy) cyclopentyl ether or 4-hydroxytestosterone (1′-methoxy) cyclopentyl ether was orally administered in sesame oil to rats. The data collected for myogenic activity consisted of levator ani muscle weight and anabolic index. Anabolic index was calculated as the ratio of the increase in levator ani weight (milligram over the control) to the increase in ventral prostrate weight. The data collected for androgenic activity consisted of seminal vesicle and ventral prostrate organ weight. Ether modified 4-hydroxytestosterone exhibited a potent anabolic activity with the second highest anabolic index of (0.78) out of 38 hormones tested. The control utilized to gage potency was the oral anabolic steroid methyltestosterone which resulted in an anabolic index of (0.37). Seminal vesicle and ventral prostrate organ weight for 4-hydroxytestosterone did not result in any significant increases. This finding correlates with the previous findings which demonstrated 4-hydroxytestosterones ability to alter testosterone metabolism by inhibiting the formation of 5-alpha dihydrotestosterone (DHT). However, most other compounds with a high anabolic index resulted in a significant increase of ventral prostrate organ weight. This significant increase in organ weight is reflective of the androgenic side effects associated with steroid hormones such as benign prostrate hyperplasia.

4-hydroxytestosterone is an natural anabolic/androgenic metabolite of 4hydroxy-4-androstenedione, which is an analog of the naturally occurring dietary supplement 4-androstenedione. To the best of my knowledge, the use of 4-hydroxytestosterone for the regulation of athletic function as a dietary supplement has never been disclosed. The fact that 4-hydroxytestosterone is naturally made in the body during the metabolism of 4-hydroxy-4-androstenedione is not widely known and is justification for its use as a dietary supplement. The esters and ethers of 4-hydroxy-4-androstenedione impart an anabolic/androgenic effect and represent a novel and unobvious improvement in the regulation of athletic function. However, 4-hydroxytestosterone also exhibits unique characteristics over other androgenic/anabolic hormones wherein 4-hydroxytestosterone has the ability to impart a mild androgenic effect with a potent anabolic effect without causing hepatotoxicity, DHT accumulation, supraphysiological surges in androgen blood concentrations, and down regulation of the hypothalamic pituitary axis. 4-hydroxytestosterone represents still a further improvement over standard anabolic/androgenic therapies and also represents a novel and unobvious improvement in athletic function.

Increases in lean body mass, strength, and work performance are predominately associated with the amount of free or active testosterone available in the body. These increases contribute to the regulation of athletic function and thus lead to enhanced physical performance. The ester and ether derivatives of 4-hydroxytestosterone have been shown to impart an anabolic/androgenic effect with none of previously mentioned negative side effects associated with increases in androgen production. Thus the said compound can be given to humans either in conjunction with or without a high protein diet (1.25 to 1.8 grams protein/kilogram of body weight) and proper anaerobic training program in order to increase the variables associated with athletic function for the purpose of enhancing physical performance. Therefore this compound represents an improvement in standard dietary androgen supplementation for the regulation of athletic performance.

After an extensive review of the scientific literature regarding the anabolic/androgenic activity and the naturally occurring formation of 4-hydroxytestosterone, it then became the focus of this invention that the esters and ethers of 4-hydroxytestosterone could be administrated perorally as an effective means of enhancing physical performance in humans without causing androgen related side effects. The oral daily doses can be between 20 to 1000 mg., but preferably 100 to 600 mg. The preferred daily dosing schedule should be divided into 3-6 sub dose applications per day in order maintain adequate blood hormone concentrations. In addition to peroral use, the esters and ethers of 4-hydroxytestosterone can be effectively administered by several other routes including transdermal, sublingual, and intranasal.

1. A method of enhancing physical performance in humans by the administration of an effective amount of at least one of the ester or ether derivatives of 4-hydroxytestosterone.

2. A method of claim 1, wherein said compound is 4alpha,17beta-Dihydroxy-4-androsten-3-one.

3. A method of claim 1, wherein said compound is 4beta,17beta-Dihydroxy-4-androsten-3-one.

4. A method of claim 1, wherein said ester derivatives include acetate, heptanoate, decanoate, hemisuccinate, benzoate and propionate.

5. A method of claim 1, wherein said carbonate ester derivatives include methyl, ethyl, propyl, butyl, and cyclohexyl.

6. A method of claim 1, wherein said ether derivatives include tetrahydropyranyl, tetrahydrofuranyl, cyclopentyl, and cyclohexyl.

7. A method of claim 1, wherein said administration is peroral.

8. A method of claim 1, wherein said administration is selected from the group consisting of transdermal, sublingual, and intranasal.

9. A method of claim 1, wherein said effective amount is a daily dosage of 20 mg to 1000 mg.
